(19) United States
(12) Patent Application Publication
(10) Pub. No.: US 2019/0223790 A1
(43) Pub. Date: Jul. 25, 2019

(54) MAGNETIC RESONANCE IMAGING APPARATUS

(71) Applicant: SAMSUNG ELECTRONICS CO., LTD., Suwon-si (KR)

(72) Inventors: Se Jin YOO, Seoul (KR); Vladimar PARAMONOV, Moscow (RU); Suk Hoon OH, Suwon-si (KR)

(73) Assignee: SAMSUNG ELECTRONICS CO., LTD., Suwon-si (KR)

(21) Appl. No.: 16/329,623

(22) PCT Filed: Jun. 15, 2017
(86) PCT No.: PCT/KR2017/006256

§ 371 Date: Feb. 28, 2019

(30) Foreign Application Priority Data
Aug. 30, 2016 (KR) 10-2016-0111113

(51) Int. Cl. A61B 5/00; A61B 5/055; G06T 7/00; G06T 7/70; G06T 7/62

(52) U.S. Cl. CPC A61B 5/4887; A61B 5/055; A61B 5/743; A61B 5/7475; A61B 5/0035; G06T 2207/30016; G06T 7/70; G06T 7/62; A61B 5/0042; G06T 2207/10088; G06T 2207/30096; G06T 7/0012

(57) ABSTRACT

A magnetic resonance imaging (MRI) apparatus comprises an image processor configured to generate a cross-sectional image of an object and configured to detect a lesion contained in the cross-sectional image and a size of the lesion, and a display configured to display the cross-sectional image and a marker indicating the lesion, and the display displays the marker in the vicinity of the lesion, and when the cross-sectional image is a cross-sectional image showing the largest size of the lesion, configured to display a first color marker in the vicinity of the lesion, and when the cross-sectional image is not a cross-sectional image showing the largest size of the lesion, configured to display a second color marker in the vicinity of the lesion.

### TECHNICAL FIELD

The present disclosure relates to a magnetic resonance imaging apparatus.

### BACKGROUND ART

In general, medical imaging apparatuses are configured to acquire information on a patient and provide an image. Medical imaging apparatuses include X-ray apparatuses, ultrasonic diagnostic apparatuses, computed tomography apparatuses, and magnetic resonance imaging (MRI) apparatuses.

Among these, MRI apparatuses play an important role in fields using medical images because they have relatively free image capture condition and provide excellent contrast in soft tissues and various diagnostic information images.

Magnetic Resonance Imaging (MRI) represents an image indicating the density and the physical and chemical properties of atomic nuclei by generating nuclear magnetic resonance of hydrogen atomic nuclei in the body using Radio Frequencies (RF) as specific ionization radiation and magnetic fields that are not harmful to human bodies.

Particularly, the MRI apparatus supplies energy and a predetermined frequency while applying a constant magnetic field inside a gantry, and converts energy released from the nucleus into a signal, thereby imaging the inside of the object.

When the MRI apparatus images an object, the output image may contain an image related to lesions. Therefore, a user has visually determined the size or the change in the size of lesions of an object over time.

### DISCLOSURE

### Technical Problem

The present disclosure is directed to providing a magnetic resonance imaging apparatus capable of intuitively displaying characteristics of lesions contained in an image.

Further, the present disclosure is directed to providing a magnetic resonance imaging apparatus capable of visually displaying whether a displayed image is an image about a cross-section showing the largest size of a lesion.

Further, the present disclosure is directed to providing a magnetic resonance imaging apparatus capable of providing a statistical model for lesions contained in an image.

### Technical Solution

One aspect of the present disclosure provides a magnetic resonance imaging (MRI) apparatus including an image processor configured to generate a cross-sectional image of an object and configured to detect a lesion contained in the cross-sectional image and a size of the lesion, and a display configured to display the cross-sectional image and a marker indicating the lesion, and the display displays the marker in the vicinity of the lesion, and when the cross-sectional image is a cross-sectional image showing the largest size of the lesion, configured to display a first color marker in the vicinity of the lesion, and when the cross-sectional image is not a cross-sectional image showing the largest size of the lesion, configured to display a second color marker in the vicinity of the lesion.

The MRI apparatus may further include an inputter configured to receive a cross-section change command from a user, and the image processor may generate a plurality of cross-sectional images, and when the cross-section change command is input, the display may display other cross-sectional image of the object.

The display may display the first color marker or the second color marker in the vicinity of a lesion according to the size of the lesion contained in the other cross-sectional image.

The inputter may include a trackball or a scroll wheel, and the inputter may receive an operation of the trackball or an operation of the scroll wheel by a user, as the input of the cross-section change command.

The MRI apparatus may further include an inputter configured to receive a lesion designation command about at least one point on the cross-sectional image, from a user, and the display may display a marker in the vicinity of the designated point according to the lesion designation command.

The image processor may generate first and second cross-sectional images indicating the same cross-section of the object, in different image modes, and the display may display the first and second cross-sectional images on first and second sections, respectively.

The display may display first and second cursors synchronized with each other, on each section, respectively.

The MRI apparatus may further include an inputter configured to receive a cross-section change command from a user, and when the cross-section change command is input, the image processor may generate third and fourth cross-sectional images showing other cross-section of the object, in different image modes and the display may display third and fourth cross-sectional images, on the first and second sections, respectively.

The MRI apparatus may further include an inputter configured to receive a change command of sensitivity detecting the lesion, from a user, and the image processor may change a sensitivity detecting a lesion present in the cross-sectional image, based on the change command of sensitivity.

The MRI apparatus may further include an inputter configured to receive any one lesion selected from the lesions, and the image processor may detect any one of a diameter, volume, density and position of the selected lesion, and the display may display any one of the diameter, volume, density and position.

The image mode may include a T1-weighted image mode, a magnetic resonance angiography (MRA) mode, a susceptibility-weighted image (SWI) mode, an echo planar imaging (EPI) mode, and a T2-weighted imaging mode, and a maximum intensity projection (mIP) mode.

Another aspect of the present disclosure provides a magnetic resonance imaging (MRI) apparatus including an image processor configured to generate an image of an object and configured to detect one or more lesions contained in the image and a size of each of the lesion, and a display configured to display the image and a statistical model about the lesion contained in the image, and the image processor gives an identification number to the one or more lesions, and the statistical model is a graph on which a first axis indicates the identification number of the lesion and a second axis indicates the size of the lesion.

The size of the lesion may represent the diameter, volume or density of the lesion.

The image processor may give the identification number to one or more lesions in order of the size of the lesions.

The MRI apparatus may further include an inputter configured to receive at least one lesion selected from one or more lesions displayed on the image, and the display may display a graph item corresponding to the selected lesion, with highlight.

The MRI apparatus may further include an inputter configured to receive any one graph item selected from graph items about one or more lesions displayed on the statistical model, and the image processor identifies a first lesion corresponding to the selected graph item, and a cross-sectional image showing the largest diameter of the first lesion, and the display may display the cross-sectional image showing the largest diameter of the first lesion.

The statistical model may be a first statistical model, and the display may further display a second statistical model about a lesion contained in the image, and the second statistical model may be a graph on which a first axis indicates the size of the lesion and a second axis indicates the number of the lesion in each size.

The MRI apparatus may further include an inputter configured to receive at least one lesion selected from one or more lesions displayed on the image, and the display may display a graph item of the first statistical model and a graph item of the second statistical model corresponding to the selected lesion, with highlight.

### Advantageous Effects

A user can intuitively identify the size and the position of a lesion contained in an image.

A user can select a cross-sectional image on which a desired lesion is most clearly displayed, and thus the user can precisely recognize the number of lesions by identifying lesions contained in one or more cross-sectional images of an object.

A user can easily identify the change in the distribution of the lesion over time, by using a statistical mode of the lesion that is displayed.

### DESCRIPTION OF DRAWINGS

FIG. 1 is a schematic diagram of a magnetic resonance imaging (MRI) system.

FIGS. 2 and 3 are conceptual views showing an object having a plurality of cross-sections.

FIGS. 4 to 7 are views of a plurality of cross-sectional images corresponding to each of a plurality of image modes.

FIGS. 8 and 9 are conceptual views showing the plurality of cross-sectional views of an object having lesions.

FIGS. 10A and 10B views showing two cross-sectional images on which a marker having a color corresponding to a diameter of a lesion, is displayed.

FIGS. 11A and 11B are views showing a screen of an output portion displaying a marker according to designation or cancellation of a lesion by a user.

FIGS. 12 and 13 are views showing an estimated value of a lesion in a variety of forms.

FIG. 14 is a view showing a screen that is output when lesion detection sensitivity is adjusted.

FIGS. 15A to 16 are two views of statistical models for lesion diameter.

FIGS. 17 and 18 are views of statistical models showing a temporal change of the diameter distribution of the plurality of lesions.

### MODES FOR THE INVENTION

In the following description, like reference numerals refer to like elements throughout the specification. Well-known functions or constructions are not described in detail since they would obscure the one or more exemplar embodiments with unnecessary detail. Terms such as “unit”, “module”, “member”, and “block” may be embodied as hardware or software. According to embodiments, a plurality of “unit”, “module”, “member”, and “block” may be implemented as a single component or a single “unit”, “module”, “member”, and “block” may include a plurality of components.

Also, when a part “includes” or “comprises” an element, unless there is a particular description contrary thereto, the part may further include other elements, not excluding the other elements.

It will be understood that, although the terms first, second, third, etc., may be used herein to describe various elements, but is should not be limited by these terms. These terms are only used to distinguish one element from another element.

As used herein, the singular forms “a,” “an” and “the” are intended to include the plural forms as well, unless the context clearly indicates otherwise.

Throughout the description, the term “image” refers to multi-dimensional data composed of discrete image elements (e.g., pixels in two-dimensional image and voxels in a three-dimensional image). For example, images may include medical images of an object acquired by X-ray apparatuses, computed tomography apparatuses, magnetic resonance imaging (MRI) apparatuses, ultrasound and other medical imaging systems.

“Object” may include human, animals or a part thereof. For example, the object may include the organ such as the liver, the heart, the uterus, the brain, the breast, and the abdomen, or the blood vessels. “Object” may include phantom. The phantom represents a material having an approximate volume that is very close to the density and effective number of living organisms. Therefore, the phantom may include a sphere phantom having the property similar with the body.

Further, throughout the description, the term “user” refers to medical experts such as a physician, a nurse, a medical laboratory technologist, or a medical imaging expert and further refers to a technician repairing a medical device, but is not limited thereto.

The MRI system may acquire a magnetic resonance (MR) signal, and reconstruct the acquired MR signal into an image. The MR signal may represent a radio frequency (RF) signal irradiated from an object.

As for the MRI system, a main magnet may form a static magnetic field, and align the direction of the magnetic dipole moments of certain atomic nuclei in an object that is placed in the static magnetic field, as the direction of the static magnetic field. A gradient magnetic coil may apply a gradient signal to the static magnetic field and form a gradient magnetic field so as to derive a different resonance frequency for each a part of the object.

RF coils may irradiate a RF signal in accordance with a resonance frequency of a part that is to be a subject to acquire an image. In addition, as the gradient magnetic field forms, the RF coil may receive magnetic resonance signals of different resonance frequency that is emitted from a variety of parts of the object. Accordingly, the MRI system acquires an image based on the magnetic resonance signal using an image reconstruction method.

Reference will now be made in detail to embodiments of the present disclosure, examples of which are illustrated in the accompanying drawings.

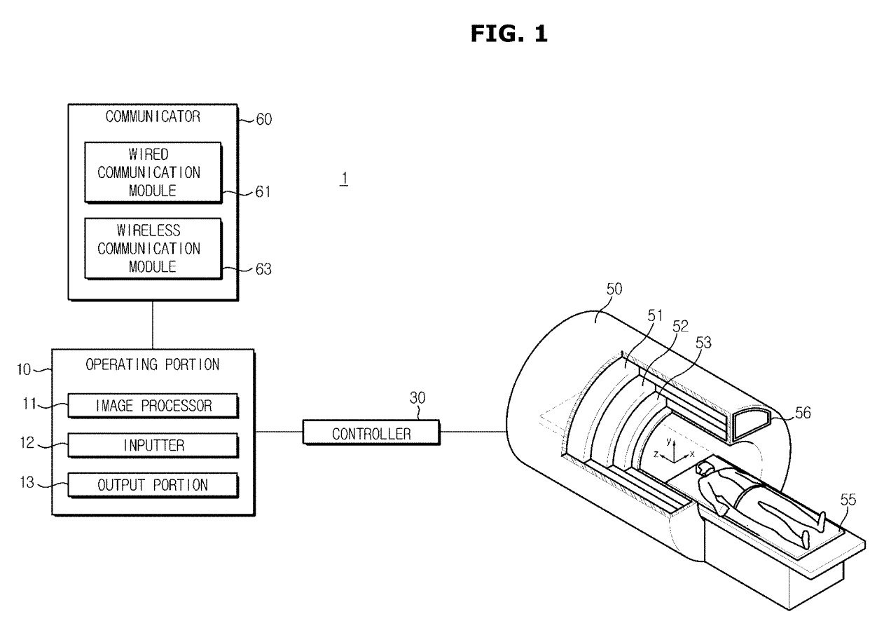

FIG. 1 is a schematic diagram of a magnetic resonance imaging (MRI) system. Referring to FIG. 1, a MRI system 1 may include an operating portion 10, a controller 30 and a scanner 50. The controller 30 may be independently implemented as illustrated in FIG. 1. Alternatively, the controller 30 may be divided into a plurality of components and then contained in each component of the MRI system 1. Hereinafter each component thereof will be described in detail.

The scanner 50 may be formed in a shape (e.g., a bore shape) in which a void is provided to allow an object to be inserted. In the inner space of the scanner 50, a static field and a gradient field may be formed and an RF signal may be emitted.

The scanner 50 may include a static field coil portion 51, a gradient coil portion 52, a RF coil portion 53, a transfer table 55 and a display 56. The static field coil portion 51 may form a static magnetic field for aligning the direction of the magnetic dipole moments of atomic nuclei in an object in the direction of the static magnetic field. The static field coil portion 51 may be implemented as a permanent magnet or a superconducting magnet using a cooling coil.

The gradient coil portion 52 may be connected to the controller 30. The gradient coil portion 52 may form a gradient magnetic field by applying a gradient to the static magnetic field according to a control signal transmitted from the controller 30. The gradient coil portion 52 may include X, Y, and Z coils that form mutually orthogonal X, Y, and Z-axis gradient magnetic fields. The gradient coil portion 52 may generate a gradient signal corresponding to an imaging position so as to differently derive resonance frequencies according to a part of the object.

The RF coil portion 53 may be connected to the controller 30 to radiate an RF signal to an object according to the control signal transmitted from the controller 30. The RF coil portion 53 may receive a MR signal emitted from the object. The RF coil portion 53 may transmit a RF signal having a frequency equal to the frequency of the precession, to the target atomic nuclei in the precession, and then stop the transmission of the RF signal, and receive a MR signal emitted from the object.

The RF coil portion 53 may be implemented with a transmission RF coil generating an electromagnetic wave having a radio frequency corresponding to the type of the atomic nuclei, and a RF reception coil receiving electromagnetic waves radiated from the atomic nuclei, or implemented with a single RF transmission/reception coil having a transmission/reception function. Other than the RF coil portion 53, an additional coil may be mounted to the object. For example, an additional coil such as a head coil, a spine coil, a torso coil, and a knee coil may be used according to an imaging part and a mounting part.

The display 56 may be provided inside and/or outside of the scanner 50. The display 56 may be controlled by the controller 30 and thus provide information related to the medical imaging to the user or the object.

In addition, the scanner 50 may include an object monitoring information acquisition portion configured to acquire and transmit monitoring information related to an object status. For example, the object monitoring information acquisition portion (not shown) may acquire monitoring information related to the object from a camera (not shown) for imaging the movement and position of the object, a breathing meter (not shown) for measuring the respiration of the object, an electrocardiogram (ECG) measuring instrument (not shown) for measuring the electrocardiogram of the object, or a body temperature measuring instrument (not shown) for measuring a temperature of the object. The object monitoring information acquisition portion (not shown) may transmit the monitoring information to the controller 30. Accordingly, the controller 30 may control the operation of the scanner 50 using the monitoring information about the object. Hereinafter, the controller 30 will be described.

The controller 30 may control the overall operation of the scanner 50.

The controller 30 may control the sequence of signals formed within the scanner 50. The controller 30 may control the gradient coil portion 52 and the RF coil portion 53 according to the pulse sequence received from the operating portion 10 or a designed pulse sequence.

Pulse sequence includes all kinds of information for controlling the gradient coil portion 52 and the RF coil portion 53, e.g., an intensity of pulse signal, a duration of application, and an application timing, wherein the pulse signal is applied to the gradient coil portion 52.

The controller 30 may control the gradient magnetic field generation of the gradient coil portion 52 by controlling a waveform generator (not shown) generating a gradient waveform, i.e., a current pulse, according to a pulse sequence, and a gradient amplifier (not shown) amplifying the generated current pulse and transmitting the amplified current pulse to the gradient coil portion 52.

The controller 30 may control the operation of the RF coil portion 53. For example, the controller 30 may supply an RF pulse having a resonance frequency to the RF coil portion 53 so as to irradiate the RF signal, and the controller 30 may receive the MR signal received by the RF coil portion 53. In this time, the controller 30 may control an operation of switch (e.g.,T/R switch), which is configured to change the transmission and reception direction, by a control signal so as to control the irradiation of the RF signal and the reception of the MR signal according to an operation mode.

The controller 30 may control the movement of the transfer table 55 in which the object is located. Before the imaging is performed, the controller 30 may move the transfer table 55, in accordance with a target part of the object.

The controller 30 may control the display 56. For example, the controller 30 may control ON/OFF state of the display 56 or a screen displayed on the display 56.

The controller 30 may be implemented using a memory (not shown) storing an algorithm for controlling an operation of components in the MRI system 1 and data related to programs implementing the algorithm, and a processor (not shown) performing the above mentioned operation using the data stored in the memory. The memory and the processor may be implemented in separate chips, or a single chip.

The operating portion 10 may control the overall operation of the MRI system 1. The operating portion 10 may include an image processor 11, an inputter 12, and an output portion 13.

The image processor 11 may be implemented using a memory (not shown) storing an algorithm for controlling an operation of components in the operating portion 10 and data related to programs implementing the algorithm, and a processor (not shown) performing the above mentioned operation using the data stored in the memory. The memory and the processor may be implemented in separate chips, or a single chip.

The image processor 11 may store the MR signal received from the controller 30 by using the memory, and generate an image data about the object, from the stored MR signal by applying the image reconstruction technique by using the processor.

For example, when a k-space data is completed by filling digital data in a k-space of the memory (referred to as a Fourier space or a frequency space), the image processor 11 may restore the k-space data to image data by applying various image restoration techniques (e.g., by performing inverse Fourier transform on the k-space data) by using the processor.

Image restoration techniques may include various techniques that restores an image using various well-known MR image mode such as a T1-weighted image mode, a magnetic resonance angiography (MRA) mode, a susceptibility-weighted image (SWI) mode, an echo planar imaging (EPI) mode, and a T2-weighted imaging mode, and a maximum intensity projection (mIP) mode.

In addition, various signal processes applied to the MR signal by the image processor 11 may be performed in parallel. For example, the image processor 11 may perform a signal processing on a plurality of MR signals, which is received by a multiple RF coil, in parallel, so as to restore images. Further, the image processor 11 may store the restored image in a memory or, as will be described later, the controller 30 may store the stored image in an external server through a communicator 60.

The image processor 11 according to one embodiment may store the generated image by using the memory, and detect lesions contained in the image by using the processor, thereby generating an estimated value of lesions of the object based on the stored image.

The estimated value of lesions may be various measured values related to lesions. For example, the estimated value of lesions may include a size of the lesion contained in the image, such as a diameter, a volume and a density, a position of lesion, or an identification number of lesion. A method of automatically detecting lesion may be detecting a dark point region in an image.

The image processor 11 according to one embodiment may store an estimated value of lesion by using the memory, and generate a statistical model related to the diameter, volume or density of the lesion by using the processor. A description related to the statistical model will be described later.

When the inputter 12 receives a change command of sensitivity for detecting a lesion, from a user, the image processor 11 according to one embodiment may change the sensitivity for detecting a lesion present in the image, based on the change command of sensitivity.

In addition, according to one embodiment the image processor 11 may control overall output portion 13.

The image processor 11 according to one embodiment may store the generated estimated value of lesion by using the memory, and control the output portion 13 to display a marker in a color corresponding to a diameter of a lesion by using the processor.

When the inputter 12 receives a cross-section change command while the output portion 13 outputs any one cross-sectional image of the object, the image processor 11 according to one embodiment may control the output portion 13 so that the output portion 13 outputs a cross-sectional image about other cross-section of the object.

When the inputter 12 receives a lesion destination command while the output portion 13 outputs any one cross-sectional image of the object, the image processor 11 according to one embodiment may control the output portion 13 so that the output portion 13 displays a marker in the vicinity of a point designated by the lesion destination command.

When generating first and second cross-sectional images indicating the same cross-section of the object, in different image modes, the image processor 11 according to one embodiment may control the output portion 13 so that the output portion 13 displays the first and second cross-sectional image on a first and second section of the screen, respectively. In this case, the image processor 11 may allow the output portion 13 to display first and second cursors which are synchronized with each other, on each section.

When the inputter 12 receives the cross-section change command from a user while the first and second cross-sectional image are output on the output portion 13, the image processor 11 according to one embodiment may generate third and fourth cross-sectional images indicating other cross-section of the object, in different image modes, and control the output portion 13 so that the output portion 13 displays the third and fourth cross-sectional images on the first and second sections, respectively.

When the inputter 12 receive any one lesion selected from the plurality of lesions displayed by the output portion 13, the image processor 11 according to one embodiment may detect at least one of diameter, volume, density and position of the selected lesion, and control the output portion 13 so that the output portion 13 displays at least one of the detected diameter, volume, density and position.

A detailed operation process of the image processor 11 will be described later.

The inputter 12 may receive a control command about the overall operation of the MRI system 1 from a user. For example, the inputter 12 may receive object information, parameter information, scan conditions, and information on pulse sequences from a user. The inputter 12 may be implemented as a keyboard, a mouse, a trackball, a voice recognition portion, a gesture recognition portion, or a touch screen.

The output portion 13 may output an image generated by the image processor 11. Further, the output portion 13 may output a user interface (UI) configured to allow a user to input a control command related to the MRI system 1. The output portion 13 may be implemented with a speaker, a printer, or a display. The display may include the display 56 provided outside and/or inside the scanner 50. The embodiments described below will illustrate that the output portion 13 is implemented as a display, but is not limited thereto.

The display may be implemented as cathode-ray tube (CRT), digital light processing (DLP) panel, plasma display panel, liquid crystal display (LCD) panel, electro luminescence (EL) panel, electrophoretic display (EPD) panel, Electrochromic Display (ECD) panel, light emitting diode (LED) panel or organic light emitting diode (OLED) panel, but is not limited thereto.

Meanwhile, FIG. 1 shows that the operating portion 10 and the controller 30 are separated from each other, but the operating portion 10 and the controller 30 may be contained in a single apparatus, as mentioned above. Also, the processes performed by each of the operating portion 10 and the controller 30 may be performed by different components. For example, the image processor 11 may convert an MR signal received by the controller 30 into a digital signal or the controller 30 may convert an MR signal into a digital signal by itself.

In accordance with the performance of the components of the MRI system 1 shown in FIG. 1, at least one component may be added or deleted. In addition, the mutual position of the components may be changed in accordance with the performance or structure of the system, which is appreciated by those skilled in the art.

Each component shown in FIG. 1 may represent a software element or a hardware element, such as a Field Programmable Gate Array (FPGA) or an Application Specific Integrated Circuit (ASIC).

Hereinafter a detail process of outputting a marker, which is in a color corresponding to the size of lesion, with an image, and outputting a plurality of images corresponding to respective image modes when the MRI system 1 according to one embodiment outputs an image, will be described.

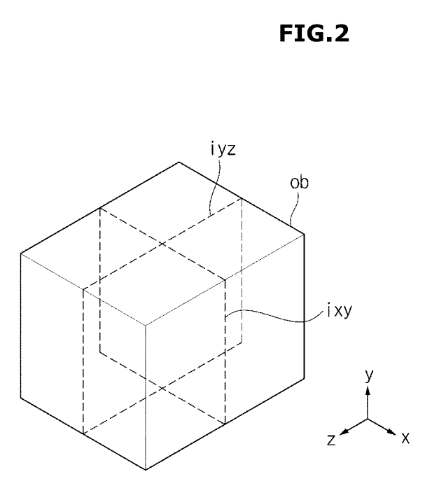

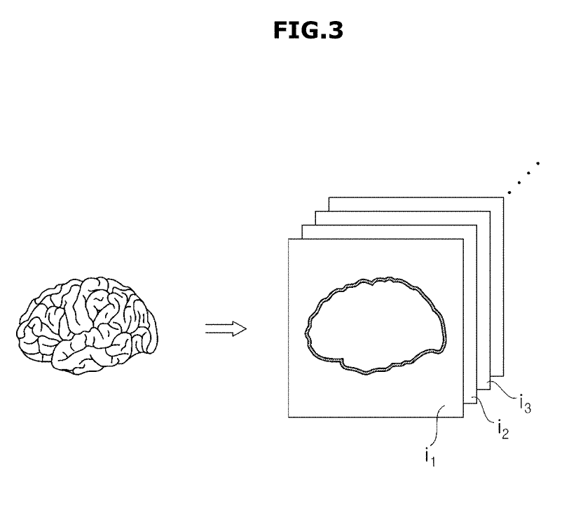

FIGS. 2 and 3 are conceptual views showing an object having a plurality of cross-sections.

As described above, the object ob may include human, animals or a part thereof. For example, the object may include the organ such as the liver, the heart, the uterus, the brain, the breast, and the abdomen, or the blood vessels, or phantom. The object ob may be represented as three-dimensional shape having a volume, and may include one or more cross-sections ixy and iyz, as illustrated in FIG. 2. The cross-section ixy and iyz may represent a slice of the object ob which is shown when slicing the object ob in various directions (e.g., xy plane, yz plane, and xz plane). The planes in various directions are shown in FIG. 2 are not limited to xy plane, yz plane, and xz plane.

When the object ob is part of the body, the object ob may include not only normal cell tissues but also a lesion such as microhemorrhage. Hereinafter an example in which the object ob is the brain will be described.

Referring to FIG. 3, the output portion 13 of the MRI system 1 according to one embodiment may output a plurality of cross-sectional images i1, i2, and i3.

The plurality of cross-sectional images i1, i2, and i3 is a plurality of 2D images about a plurality of cross-sections which is shown when slicing the object ob by many times in any one direction. For example, the plurality of cross-sectional images i1, i2, and i3 may be a plurality of 2D images about a plurality of cross-sections which is shown when slicing the brain along the z-axis direction in the xy plane of FIG. 2.

The plurality of cross-sectional images i1, i2, and i3 may construct a 3D image of any one object ob.

The output portion 13 according to one embodiment may simultaneously output the plurality of cross-sectional images i1, i2, and i3 on a single screen. Alternatively, the output portion 13 may output a single cross-sectional image on a single screen, and particularly, the output portion 13 may output other cross-sectional image of other section of the object ob according to an operation of the inputter 12 (i.e., input of cross-section change command).

The cross-section change command may be a scroll operation or a wheel operation of the inputter 12.

In addition, the output portion 13 may output a plurality of cross-sectional images corresponding to each of the plurality of image mode, about any one cross-section of the object, based on the image data of the image processor 11, on a single screen.

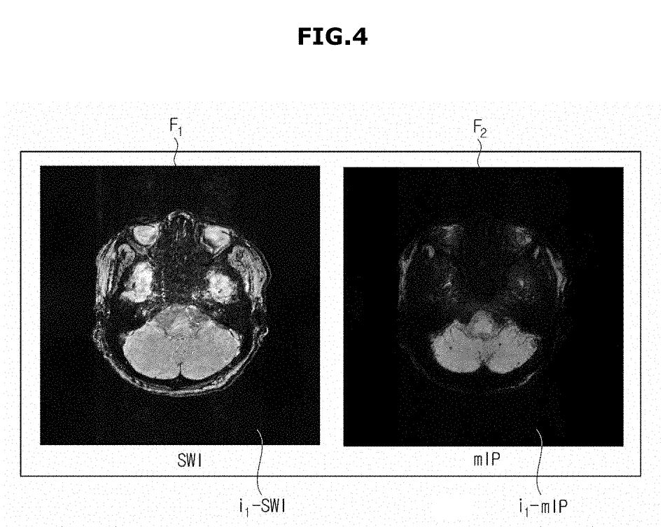

FIGS. 4 to 7 are views of a plurality of cross-sectional images corresponding to each of a plurality of image modes.

Referring to FIG. 4, a screen output by the output portion 13 may include a plurality of sections F1 and F2. The output portion 13 may display a plurality of cross-sectional images i1-SWI and i1-mIP corresponding to each of the plurality of image modes, on the plurality of sections F1 and F2. The plurality of cross-sectional images i1-SW1 and i1-mIP may be first and second cross-sectional images about the same cross-section of the object ob.

For example, the output section 13 may display the first cross-sectional image it-SWI generated in the SWI mode, on the left section F1 on the screen, and the second cross-sectional image i1-mIP generated in the mIP mode, on the right section F2 on the screen.

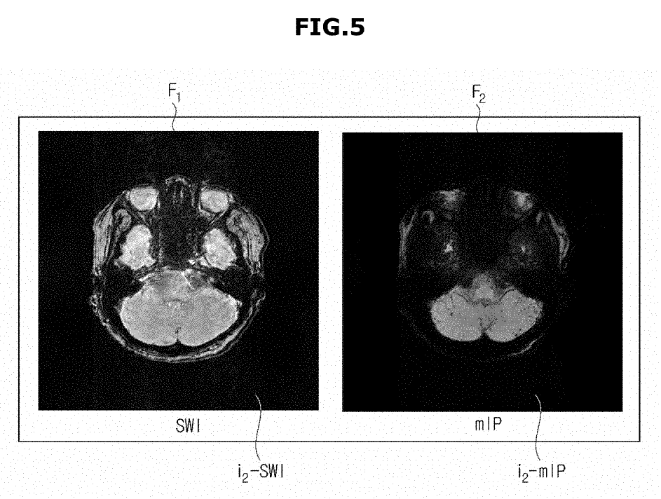

As illustrated in FIG. 4, when the inputter 12 receives the cross-section change command while the output portion 13 outputs a plurality of cross-sectional images corresponding to each of the plurality of image mode, about any one cross-section of the object, on a single screen, the output portion 13 may output a plurality of cross-sectional images i2-SW1 and i2-mIP about other cross-section of the object ob, as illustrated in FIG. 5.

In this case, the plurality of cross-sectional images i2-SW1 and i2-mIP about other cross-section may be generated in the same image mode as the plurality of cross-sectional images i1-SW1 and i1-mIP that is about any one cross-section before inputting the cross-section change command.

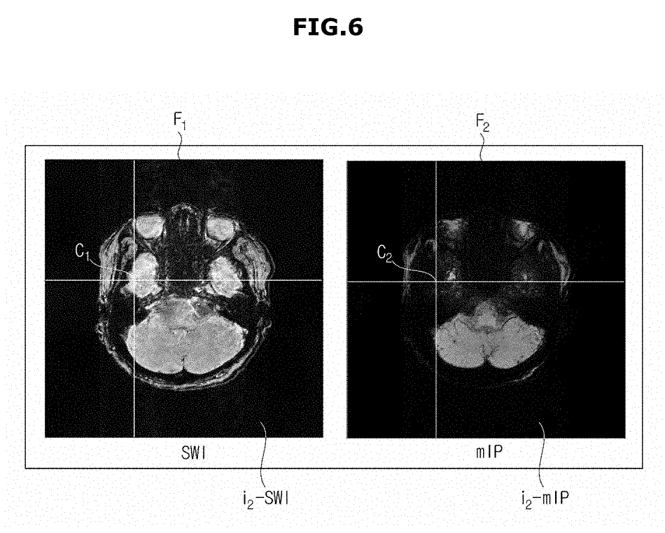

Further, referring to FIG. 6, the output portion 13 according to one embodiment may display the cursor C1 and C2, which are synchronized with each other, together with the cross-sectional images i2-SW1 and i2-mIP on each section F1 and F2 on the screen.

When the inputter 12 is implemented with a mouse, the cursor may be moved according to the movement of the mouse. When a user moves the cursor C1 on any one section F1 on the screen, the image processor 11 may control the cursor C2 displayed on other section F2 to be moved in the same direction as the cursor C1.

When the synchronized cursors C1 and C2 are displayed, a user can easily identify that any one point on the cross-sectional image i2-SWI on the one section F1 corresponds to which point on the other section F2.

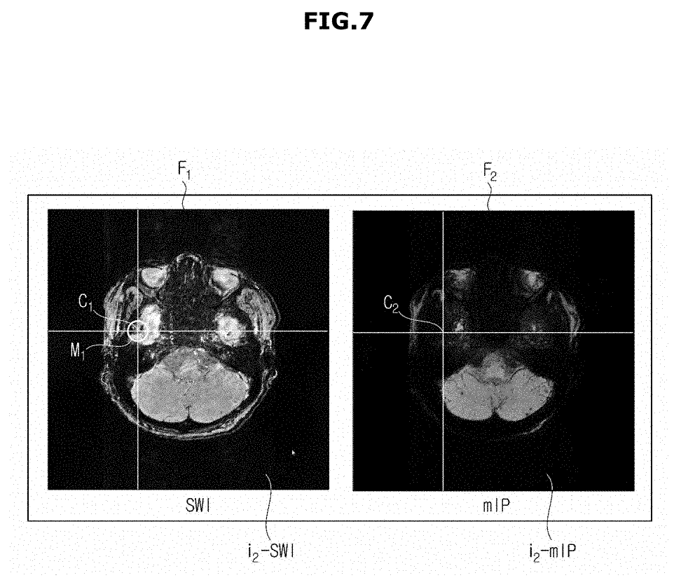

Further, referring to FIG. 7, the inputter 12 according to one embodiment may select any one point, to which the cursor C1 is directed, as a lesion, and in this case, the output portion 13 may display a marker M1 in the vicinity of the selected point according to a lesion selection command. The output portion 13 may display a marker in a color corresponding to the diameter of the lesion that is calculated by the image processor 11. For example, when the cross-sectional image i2-SWI indicating the largest diameter of a lesion (i.e., passing through the center portion of the lesion) is displayed, the output portion 13 may display a red marker M1 according to a control signal of the image processor 11. When a cross-section image passing through other point then the center of the lesion is displayed, the output portion 13 may display a yellow marker (not shown) according to a control signal of the image processor 11. The change of the graphic attributes such as the color or shape of the marker M1 is not limited thereto.

On the other hand, as described above, the lesion of the object ob may be automatically detected by the image processor 11, as well as manually detected by the user. In this case, the output portion 13 according to one embodiment may display a marker in a color corresponding to the size of the detected lesion.

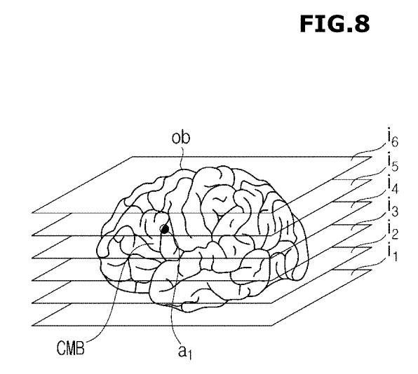

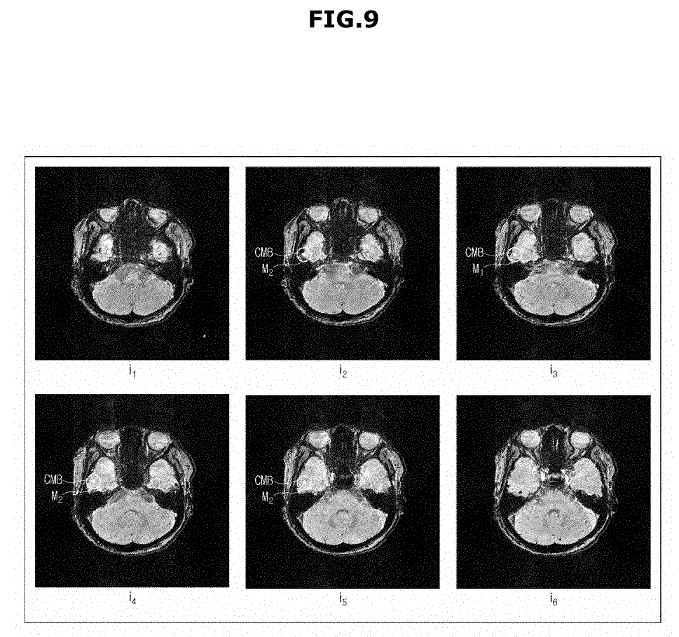

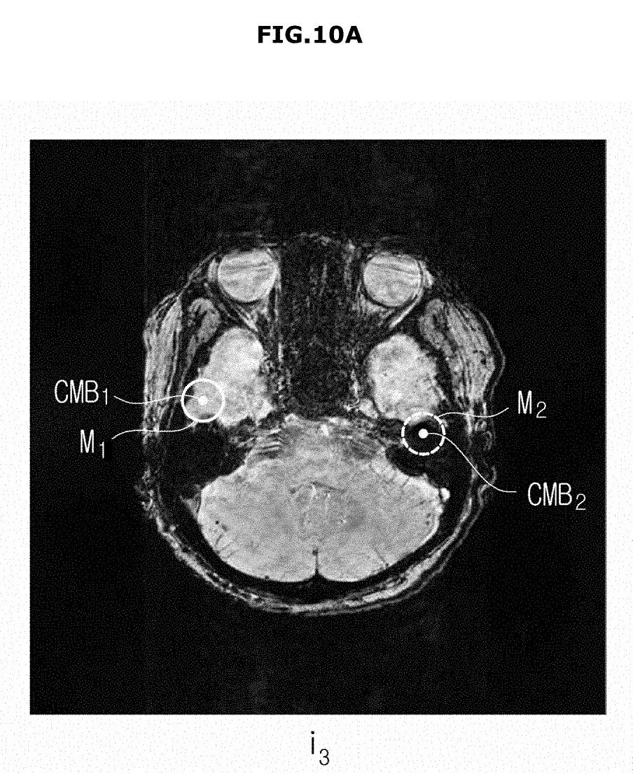

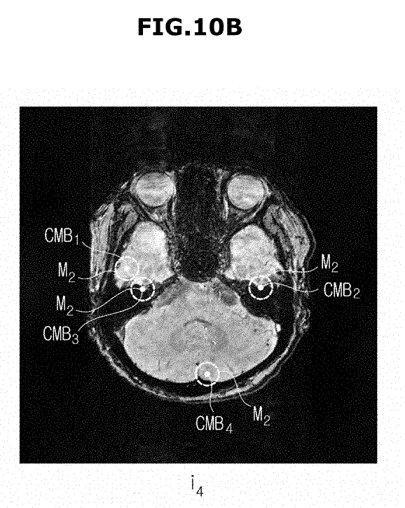

FIGS. 8 and 9 are conceptual views showing the plurality of cross-sectional views of an object having lesions, and FIGS. 10A and 10B views showing two cross-sectional images on which a marker having a color corresponding to a diameter of a lesion, is displayed.

Referring to FIG. 8, the lesion (i.e., Cerebral microbleeds; CMB) of the object ob may have a volume. The lesion CMB may be detected on cross-sectional images i2 to i5 of some part of object ob, but the lesion CMB may be not detected on cross-section images i1 and i6.

In addition, the lesion CMB generally has the largest diameter at the central portion al. Accordingly, the image processor 11 of the MRI system 1 may identify a cross-sectional image i3 having the largest diameter of the lesion CMB among the cross-sectional images i2 to i5 on which the lesion CMB is detected, and estimate the corresponding cross-sectional image i3, as the cross-sectional image passing through the center portion al of the lesion CMB.

Further, referring to FIG. 9, the output portion 13 according to one embodiment may not display the markers M2 and M1 in the vicinity of the lesion CMB on the cross-section images i1 and i6 on which the lesion CMB is not detected, but may display the markers M2 and M1 in the vicinity of the lesion CMB on the cross-section images i2 to i4 on which the lesion CMB is detected. The vicinity of the lesion CMB may include a region on which the lesion CMB is present or a position apart from the region on which the lesion CMB is present, by a predetermined distance.

The first color marker M1 may be displayed in the vicinity of the lesion CMB on the cross-sectional image i3 passing through the center portion al of the lesion CMB and the second color marker M2 may be displayed in the vicinity of the lesion CMB on the cross-sectional images i2, i4, and i5 not passing through the center portion al of the lesion CMB. For example, the first color may be red and the second color may be yellow, but is not limited thereto. In addition, FIG. 9 shows a circular marker, but the shape of marker is not limited thereto.

Referring to FIGS. 10A and 10B, while outputting the cross-sectional image i3 of FIG. 10, the output portion 13 according to one embodiment may output other cross-section image i4 of FIG. 10 of the object ob according to an operation of the inputter 12 (i.e., the cross-section change command). In this case, each of cross-sectional images i3 and i4 may display the plurality of markers M2 and M1 according to the size of the detected plurality of lesions CMB1-CMB2 of FIG. 10A and CMB1-CMB4 of FIG. 10A.

Particularly, when the cross-section image i3 of FIG. 10A is output, and when the cross-section image i3 passes through the center portion of a first lesion CMB1, but does not pass through the center portion of a second lesion CMB2, the output portion 13 may display the first color marker M1 in the vicinity of the first lesion CMB1, and display the second color marker M2 in the vicinity of the second lesion CMB2.

However, when the cross-section image i4 of FIG. 10B is output according to the cross-section change command, and when the cross-section image i4 does not pass through the center portion of the first lesion CMB1, the second lesion CMB2, a third lesion CMB3, and a fourth lesion CMB4, the output portion 13 may display the second color marker M2 in the vicinity of the first to fourth lesions CMB1 to CMB4.

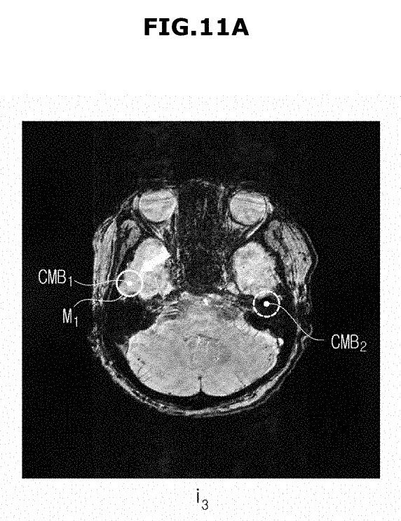

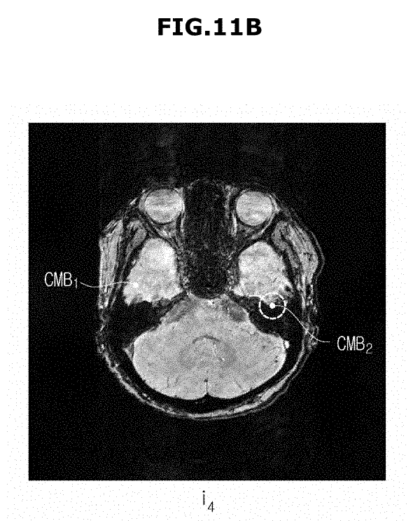

On the other hand, as for the lesion CMB that is not detected by the image processor 11, the MRI system 1 according to one embodiment may manually receive a designation of the lesion CMB via the inputter 12. As for the lesion CMB that is detected by the image processor 11, the MRI system may manually receive the detection or the cancellation of the designation of the lesion CMB via the inputter 12. FIGS. 11A and 11B are views showing a screen of an output portion displaying a marker according to designation or cancellation of a lesion by a user.

Referring to FIG. 11A, a user can move the cursor by operating the inputter 12 and designate the lesion CMB1 by selecting any one point. FIG. 11A illustrates the cursor as an arrow, but the cursor may have a shape illustrated in FIGS. 6 and 7, but is not limited thereto. When any one point is selected via the inputter 12, the output portion 13 may display the marker M1 in the vicinity of the selected point.

In addition, Referring to FIG. 11B, by operating the inputter 12, the user cancel the detection or the designation of the lesion CMB1, which is already detected or designated. When the detection or the designation of the lesion CMB1 is canceled via the inputter 12, the output portion may delete the marker M1 that is displayed in the vicinity of the selected point.

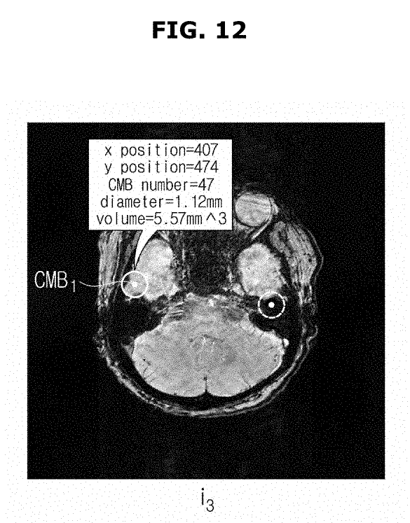

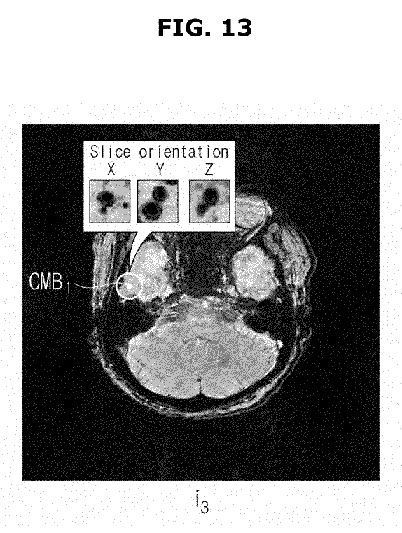

Further, the output portion 13 may display other estimated values other than the diameter of the lesion CMB. FIGS. 12 and 13 are views showing an estimated value in a variety of forms.

Referring to FIG. 12, the user may input an estimated value generation command about at least one lesion CMB1 by operating the inputter 12. When the estimated value generation command about at least one lesion CMB1 is input via the inputter 12, the output portion 13 may output a position of the selected lesion CMB1 on the cross-sectional image i3, and the identification number, diameter and volume of the lesion CMB1. The identification number of the lesion CMB1 may be randomly selected by the image processor 11 according to the number of the lesions detected in the object ob.

Further, referring to FIG. 13, the output portion 13 may output the cross-sectional image i3 in various direction (e.g., x-axis, y-axis, and z-axis direction) on which the selected lesion CMB is detected.

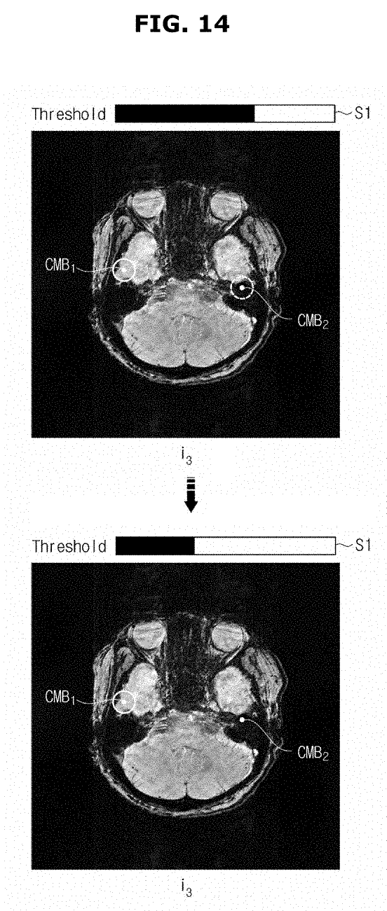

Further, when the image processor 11 automatically detects a lesion, the user may adjust a lesion detection sensitivity by operating the inputter 12. FIG. 14 is a view showing a screen that is output when lesion detection sensitivity is adjusted.

For example, on the cross-sectional image i3, the image processor 11 may detect a point in which a brightness value is less than a threshold, as the lesion CMB1 and CMB2. When the user reduces the lesion detection sensitivity, i.e., reducing a reference value S1, the image processor 11 may identify that the lesion CMB2, in which its brightness value is less than the reference value before changing but its brightness value become equal to or greater than the reference value after change, is no longer the lesion.

Accordingly, the output portion 13 may display a marker in the vicinity of only the point that is detected as the lesion.

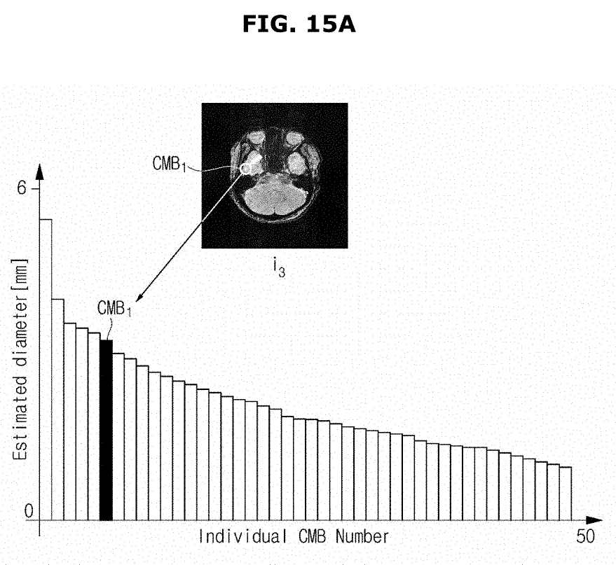

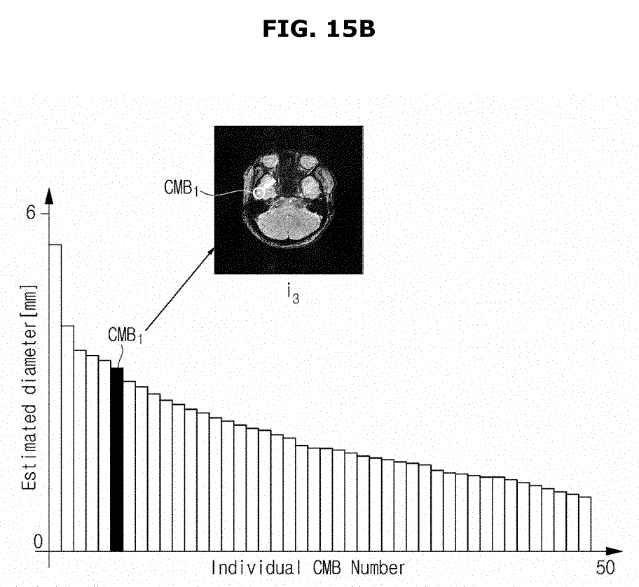

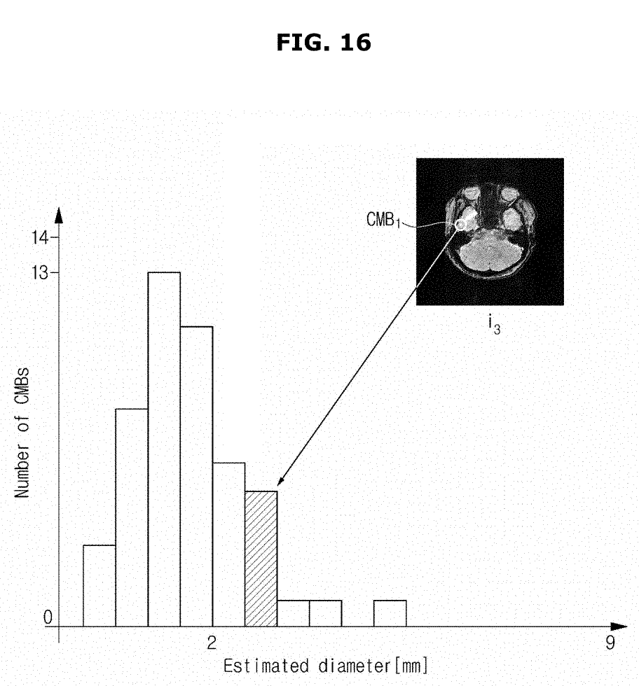

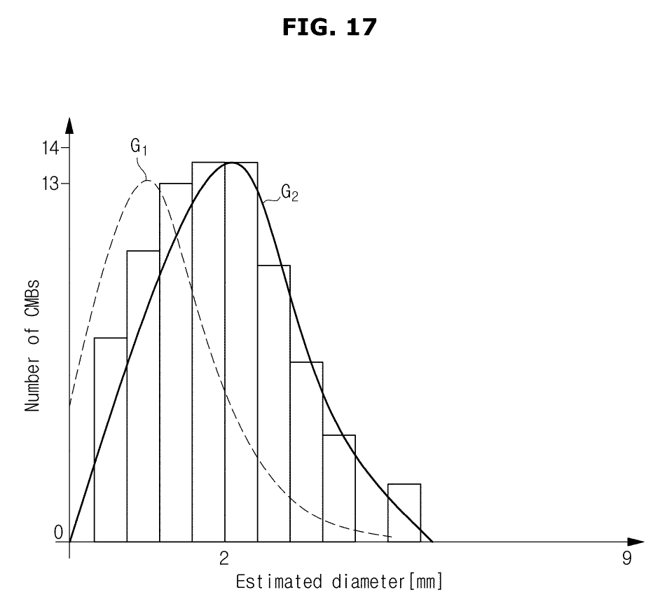

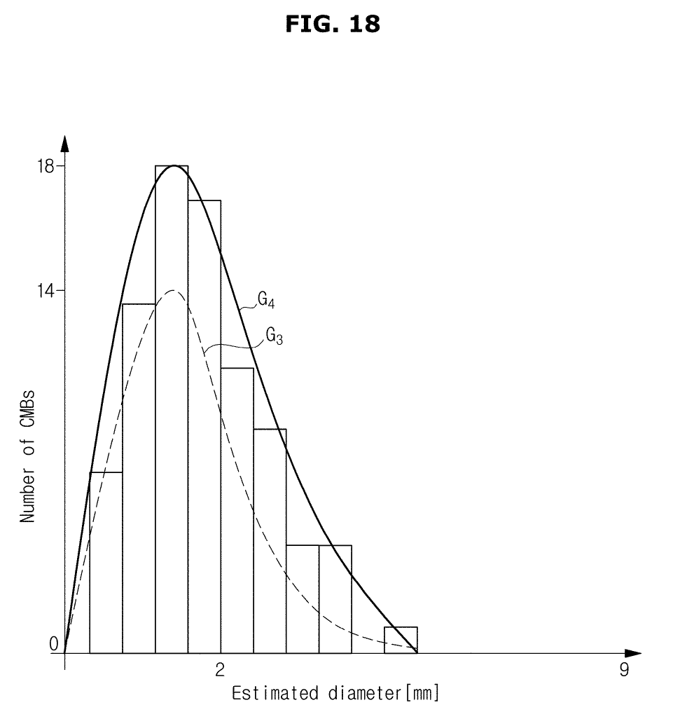

The image processor 11 according to one embodiment may store the generated estimated value about the lesion by using the memory, and generate a statistical data about the diameter or volume of the lesion by using the processor. FIGS. 15A to 16 are two views of statistical models for lesion diameter, and FIGS. 17 and 18 are views of statistical models showing a temporal change of the diameter distribution of the plurality of lesions.

The image processor 11 according to one embodiment may detect one or more lesions contained in the object ob by using the processor, give an identification number to the lesion, generate statistical data by mapping the identification number of the lesion with the maximum diameter of the lesion (i.e., the maximum diameter of the lesion on a cross-sectional image passing through the center portion of the lesion), and store the mapped statistical data by using the memory. For example, the identification number of the lesion may is given from a lesion having the maximum diameter to a lesion having the minimum diameter, but is not limited thereto.

In this case, the output portion 13 according to one embodiment may display a statistical model in the form of a graph based on the statistical data generated by the image processor 11. In this case, the horizontal axis may indicate the identification number of the lesion, and the vertical axis may indicate the diameter of the lesion.

Referring to FIGS. 15A to 15B, the output portion 13 may display a bar graph, on which the maximum diameter for each identification number is indicated by a length, as a first statistical model. Accordingly, one or more bar graphs may be arranged from a bar graph of the maximum diameter of the lesion to a bar graph of the minimum diameter of the lesion.

In this case, referring to FIG. 15A, the user may select a graph item (i.e., bar graph) of any one lesion CMB1 from the first statistical model via the inputter 12, and the output portion 13 may output a cross-sectional image i3 passing through the center portion of the selected lesion CMB1 under the control of the image processor 11. The first color marker may be displayed in the vicinity of the selected lesion CMB1 on the cross-sectional image passing through the center portion of the selected lesion CMB1.

Further, referring to FIG. 15B, the user may select any one lesion CMB1 on the cross-sectional image i3 via the inputter 12, and the image processor 11 may identify a graph item corresponding to the selected lesion CMB1 and display the graph item corresponding to the selected lesion CMB1 on the first statistical model, with highlight.

When such a first statistical model is displayed, the user can easily recognize the size distribution of diameters for the plurality of lesions.

In addition, the output portion 13 according to one embodiment may further display a second statistical model that is different from the first statistical model related to FIGS. 15A and 15B. In the second statistical model, the horizontal axis may indicate the maximum diameter of the lesion (i.e., the maximum diameter of the lesion on the cross-sectional image passing through the center portion of the lesion), and the vertical axis may indicate the number of lesions having the maximum diameter.

Referring to FIG. 16, the output portion 13 may display one or more bar graphs indicating the number of lesions having the maximum diameter as a length, as the second statistical model.

In the same as the first statistical model, the user may select any one lesion CMB1 on the cross-sectional image i3 via the inputter 12, and the image processor 11 may identify a graph item corresponding to the selected lesion CMB1 and display the graph item corresponding to the selected lesion CMB1 on the second statistical model, with highlight.

As the second statistical model is displayed, the distribution of the number of the lesions according to the maximum diameter may be displayed, and the user can determine the temporal change of the lesion based on the change of the distribution of the number of the lesions over time.

For example, as shown in FIG. 17, when the distribution of the number of the lesions according to the maximum diameter is increased in the horizontal axis direction (G1->G2), the user may determine that the size of one or more lesions contained in the object ob is generally increased.

In addition, as shown in FIG. 18, when the distribution of the number of the lesions according to the maximum diameter is increased in the vertical axis direction (G3->G4), the user may determine that the number of the lesions is increased.

The above-mentioned example of the first and second statistical models has been described as a graph about the maximum diameter of the lesion on the cross-sectional image, but it may be implemented as a graph about other estimated value of the lesion such as a graph about a volume or density on the 3D image.

Meanwhile, the disclosed embodiments may be embodied in the form of a recording medium storing instructions executable by a computer. The instructions may be stored in the form of program code and, when executed by a processor, may generate a program module to perform the operations of the disclosed embodiments. The recording medium may be embodied as a computer-readable recording medium.

The computer-readable recording medium includes all kinds of recording media in which instructions which can be decoded by a computer are stored. For example, there may be a Read Only Memory (ROM), a Random Access Memory (RAM), a magnetic tape, a magnetic disk, a flash memory, and an optical data storage device.

While the present disclosure has been particularly described with reference to exemplary embodiments, it should be understood by those of skilled in the art that various changes in form and details may be made without departing from the spirit and scope of the present disclosure.

1. A magnetic resonance imaging (MRI) apparatus comprising:
an image processor configured to generate a cross-sectional image of an object and configured to detect a lesion contained in the cross-sectional image and a size of the lesion; and
a display configured to display the cross-sectional image and a marker indicating the lesion,
wherein the display displays the marker in the vicinity of the lesion, and when the cross-sectional image is a cross-sectional image showing the largest size of the lesion, configured to display a first color marker in the vicinity of the lesion, and when the cross-sectional image is not a cross-sectional image showing the largest size of the lesion, configured to display a second color marker in the vicinity of the lesion.

2. The MRI apparatus according to claim 1, further comprising:
an inputter configured to receive a cross-section change command from a user,
wherein the image processor generates a plurality of cross-sectional images, and when the cross-section change command is input, the display displays other cross-sectional image of the object.

3. The MRI apparatus according to claim 2, wherein the display displays the first color marker or the second color marker in the vicinity of a lesion according to the size of the lesion contained in the other cross-sectional image.

4. The MRI apparatus according to claim 2, wherein the inputter comprises a trackball or a scroll wheel, and the inputter receives an operation of the trackball or an operation of the scroll wheel by a user, as the input of the cross-section change command.

5. The MRI apparatus according to claim 1, further comprising:
an inputter configured to receive a lesion designation command about at least one point on the cross-sectional image, from a user,
wherein the display displays a marker in the vicinity of the designated point according to the lesion designation command.

6. The MRI apparatus according to claim 1, wherein the image processor generates first and second cross-sectional images indicating the same cross-section of the object, in different image modes, and
the display displays the first and second cross-sectional images on first and second sections, respectively.

7. The MRI apparatus according to claim 1, further comprising:
an inputter configured to receive a change command of sensitivity detecting the lesion, from a user,
wherein the image processor changes a sensitivity detecting a lesion present in the cross-sectional image, based on the change command of sensitivity.

8. The MRI apparatus according to claim 1, further comprising:
an inputter configured to receive any one lesion selected from the lesions,
wherein the image processor detects any one of a diameter, volume, density and position of the selected lesion, and the display displays any one of the diameter, volume, density and position.

9. A magnetic resonance imaging (MRI) apparatus comprising:
an image processor configured to generate an image of an object and configured to detect one or more lesions contained in the image and a size of each of the lesion; and
a display configured to display the image and a statistical model about the lesion contained in the image,
wherein the image processor gives an identification number to the one or more lesions, and the statistical model is a graph on which a first axis indicates the identification number of the lesion and a second axis indicates the size of the lesion.

10. The MRI apparatus according to claim 9, wherein the size of the lesion represents the diameter, volume or density of the lesion.

11. The MRI apparatus according to claim 9, wherein the image processor gives the identification number to one or more lesions in order of the size of the lesions.

12. The MRI apparatus according to claim 9, further comprising:
an inputter configured to receive at least one lesion selected from one or more lesions displayed on the image,
wherein the display displays a graph item corresponding to the selected lesion, with highlight.

13. The MRI apparatus according to claim 9, further comprising:
an inputter configured to receive any one graph item selected from graph items about one or more lesions displayed on the statistical model,
wherein the image processor identifies a first lesion corresponding to the selected graph item, and a cross-sectional image showing the largest diameter of the first lesion, and the display displays the cross-sectional image showing the largest diameter of the first lesion.

14. The MRI apparatus according to claim 9, wherein the statistical model is a first statistical model, and the display further displays a second statistical model about a lesion contained in the image,
wherein the second statistical model is a graph on which a first axis indicates the size of the lesion and a second axis indicates the number of the lesion in each size.

15. The MRI apparatus according to claim 14, further comprising:
an inputter configured to receive at least one lesion selected from one or more lesions displayed on the image,
wherein the display displays a graph item of the first statistical model and a graph item of the second statistical model corresponding to the selected lesion, with highlight.
